(19) United States
(12) Patent Application Publication
(10) Pub. No.: US 2022/0354993 A1
(43) Pub. Date: Nov. 10, 2022

(54) BIOMIMETIC THREE-DIMENSIONAL DEVICE FOR DELIVERY OF THERAPEUTIC CELLS AND METHOD OF MAKING DEVICE

(71) Applicant: Paul GATENHOLM, Riner, VA (US)

(72) Inventor: Paul GATENHOLM, Riner, VA (US)

(21) Appl. No.: 17/787,318

(22) PCT Filed: Dec. 18, 2020
(86) PCT No.: PCT/US2020/065910

§ 371 Date: Jun. 19, 2022

Related U.S. Application Data
(60) Provisional application No. 62/950,253, filed on Dec. 19, 2019.

(51) Int. Cl. A61L 27/38; A61L 27/48; A61L 27/52; A61L 27/56; C12N 5/00; B33Y 80/00; A61L 27/20

(52) U.S. Cl. CPC A61L 27/3834; A61L 27/48; A61L 27/52; A61L 27/56; C12N 5/0062; B33Y 80/00; A61L 27/20; C12N 2533/78

(57) ABSTRACT

A cell delivery device and a method of producing a three dimensional device which is vascularized when implanted or topologically applied to human or animal body. Cell laden hydrogel (cells mixed with hydrogel) is casted or injected or 3D bioprinted in a leaf-like form, which contains removable parts (templates). After crosslinking, the templates are removed and the channel for vascularization is created. The device is ready for use in vitro or in vivo.

### CROSS-REFERENCE TO RELATED APPLICATIONS

100011 The present application relies on the disclosures of and claims priority to and the benefit of the filing date of U.S. Provisional Patent Application No. 62/950,253, filed Dec. 19, 2019. The disclosures of that application are hereby incorporated by reference herein in their entireties.

### BACKGROUND OF THE INVENTION

### Field of the Invention

The present invention generally relates to cell delivery device and more particularly relates to a method of producing a three dimensional device which is vascularized when implanted or topologically applied to human or animal body. The design of the device is inspired by leaf architecture. Cell laden hydrogel (cells mixed with hydrogel) is casted or injected or 3D bioprinted in the leaf-like form which contains removable parts or components (templates). After crosslinking, the templates are removed and the channel for vascularization is created. The device is ready for use in vitro or in vivo.

### Description of Related Art

Cells produce soluble ligands or other extracellular components which can diffuse out and be used for communication with other cells. Some examples of such signalling molecules are: fibroblast growth factor (FGF), bone morphogenetic protein (BMP), transforming growth factor beta (TGFβ) and Wnt. Cells, also produce chemokines, cytokines and interleukins. Parenchymal cells such as hepatocytes or beta cells act as bioreactors producing proteins or enzymes such as insulin which diffuse out of the cells. Several soluble molecules are able to induce specific cellular responses depending on their local concentration. The examples of such components are growth factors. Lack of proteins or soluble factors is the major symptom of diseases such as chronic wounds or diabetes. There are novel cell therapies used for example donor cells. In many cases, the cells are injected into the host's blood system. The most injected cells quickly end up in the liver or lungs where they are killed by the immune system. There has been progress in cell development which holds the promise for future cell therapies. An example is induced pluripotent stem cells (iPSc), which can be derived from human skin and produced in large quantities. Stem cell therapies currently lack efficient delivery systems which could satisfy localization of the cells, cell viability, and efficacy. Cell encapsulation with, for example, alginates has been successfully used for many years. The cells are viable in alginate beads but the beads are exposed to fibrosis when implanted, which results in reduced diffusion of soluble molecules. Cells can be immobilized in hydrogels, for example, but the distance from the surrounding capsule has to be less than 200-300 micrometers in order to avoid necrosis (cell death). Vascularization would solve the problem with cell death and efficacy of the cell therapy, which is taught by the current invention.

According to the invention herein, a possible way to improve survival of a cells would be to design and create the structure which will provide a vascular tree to feed the cells in the device with nutrients and oxygen, as well as facilitate transport of soluble product, which cells produce and which are the subject of cell therapy.

Leaf architecture has inspired researchers to design vascular system in three-dimensional cell culture. Rong Fan et al. has described leaf-inspired artificial microvascular networks for three-dimensional cell culture using a microfluidic approach (1). The device fabricated was, however, very small and the process was complicated. In another publication, J. Priyadarshani et al. has described an approach to produce microscale devices (2). The process was complicated and included several steps.

Cellulose nanofibrils (CNF), which can be isolated from tunicates, in aspects, produced by bacteria or isolated from primary or secondary cell walls of plants, are 8-30 nm in diameter and can be up to a micrometer long (by way of example only). They have a hydrophilic surface, in aspects, and therefore bind water on their surfaces forming hydrogels already at low solid content (1-2%). CNF are biocompatible and therefore suitable for implantation. CNF can be combined with alginates and after crosslinking will form robust hydrogels and are thus suitable for cell delivery, as explained herein.

### SUMMARY OF THE INVENTION

The invention herein overcomes the abovementioned challenges with lack of efficient cell delivery devices by introducing a biomimetic device inspired by a leaf design, in embodiments, wherein the main channel and branches are interconnected and enable native vascularization. When used as tissue or organ in vitro the channels can be perfused by pump. In a preferred embodiment, the 3D leaf-like template with any dimensions are produced by 3D printing or micro-machining.

Cell therapies typically rely on production of soluble molecules by cells and use of these molecules to cure diseases. This is an example of a biological medicine. There are, however, no currently viable devices for sufficient delivery of cells which can keep the cells alive and enable distribution of soluble molecules into a host body. The invention described herein comprises a design of a device and fabrication of a leaf-inspired template device with removable sliding parts. Such a template can be produced by 3D Printing, injection molding, or micromachining. The size of the template can be adjusted to the size of an injury, the implantation size, and/or the number of cells needed for therapy. The selected cells may be mixed with a crosslinkable hydrogel and inserted into the template. This can be done by casting, injection, or 3D Bioprinting. After crosslinking of the hydrogel, the sliding parts may be removed and the core channel with interconnected branches may be obtained. The channel can be connected to a perfusion system or hooked up to artery or implanted. The open structure will lead to formation of a vascular tree within the device, in aspects. The device can be used for delivery of cells, stem cells, or parenchymal cells, for example. Vascularization provides the cells immobilized in the hydrogel with nutrients and oxygen and improves cell viability and at the same time enables distribution of soluble molecules produced by cells and used to cure disease. The device can be used in vitro as an organ or tissue model for testing drugs, on skin for wound treatment, or it can be implanted to deliver insulin producing beta cells.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The accompanying drawings illustrate certain aspects of embodiments of the present invention and should not be used to limit the invention. Together with the written description the drawings explain certain principles of the invention.

FIG. 1 shows a schematic diagram of an embodiment of the biomimetic leaf-inspired device with A. Open structure and B. Loop structure.

FIG. 2 shows 3D printed leaf-inspired form example with removable sliding parts (templates)

FIG. 3 shows an example of fabrication of the device, A. by casting of nanocellulose-alginate hydrogel, and B. by crosslinking and removal of sliding parts, and C. by perfusion with red colour dye.

FIG. 4 shows an embodiment of the device with adipose derived stem cells in tunicate nanocellulose/alginate hydrogel: A. casted into a template, and B. after crosslinking, removal of sliding parts and perfusion with blood.

### DETAILED DESCRIPTION OF EMBODIMENTS

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein comprise a design and fabrication of, in aspects, a leaf-inspired template to be used for fabrication of cell laden hydrogel device(s) aimed for use in vitro, on skin, and/or for implantation. The hydrogel may comprise synthetic or natural biopolymers. This creates porosity with defined architecture, which enables diffusion of nutrients and oxygen, and contributes to survival of cells. The interconnected channels become vascularized when the device is implanted. The channels can be designed as closed-loop and be used to perfuse the device when it is used in vitro as a model of a tissue or organ. The invention in this manner provides an innovative way to heal wounds and enable delivery of stem cells or parenchymal cells to treat diseases, such as Diabetes.

### Example 1 (Design of Template)

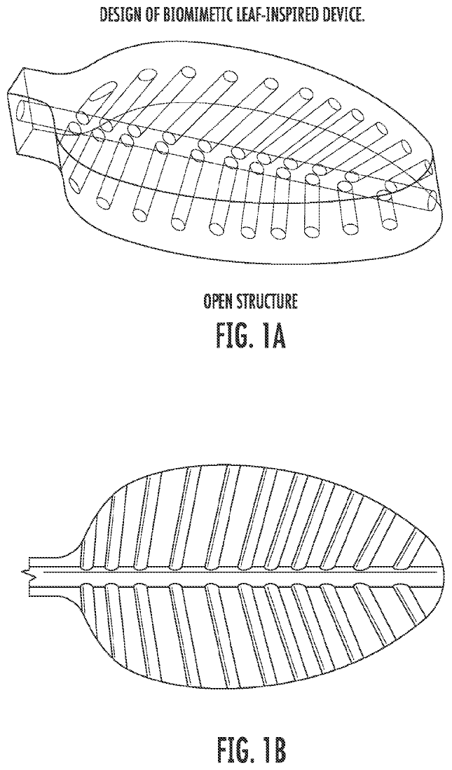

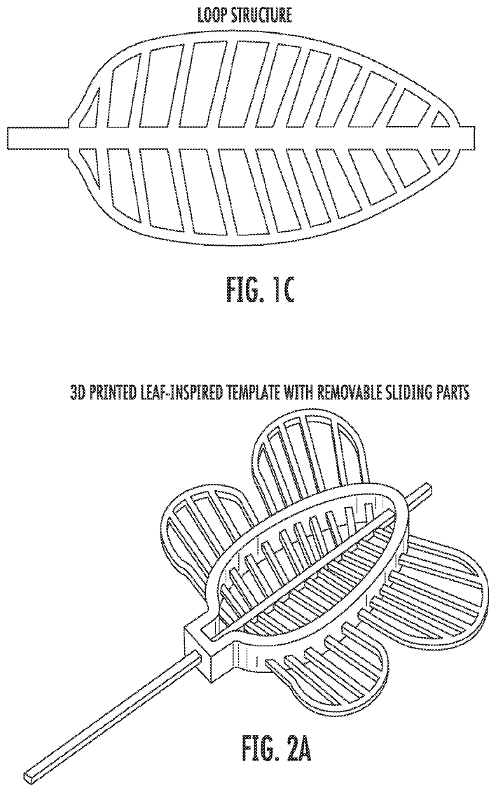

The form and templates are designed using CAD process dependent on application. The size of the leaf (length, width, thickness) is selected for an implantation site. The amount of the cells which need to be delivered affect also the size of the leaf. The architecture of the vascular tree (channels inside the leaf) is adjusted to the amount of the cells and delivery pattern which needs to be achieved. FIG. 1A shows a design of the open leaf architecture which is aimed for application on skin or for implantation. FIG. 1B shows a design of the closed-loop variety, which is aimed for in vitro use with perfusion provided by an external pump system.

### Example 2 (3D Printing of Template)

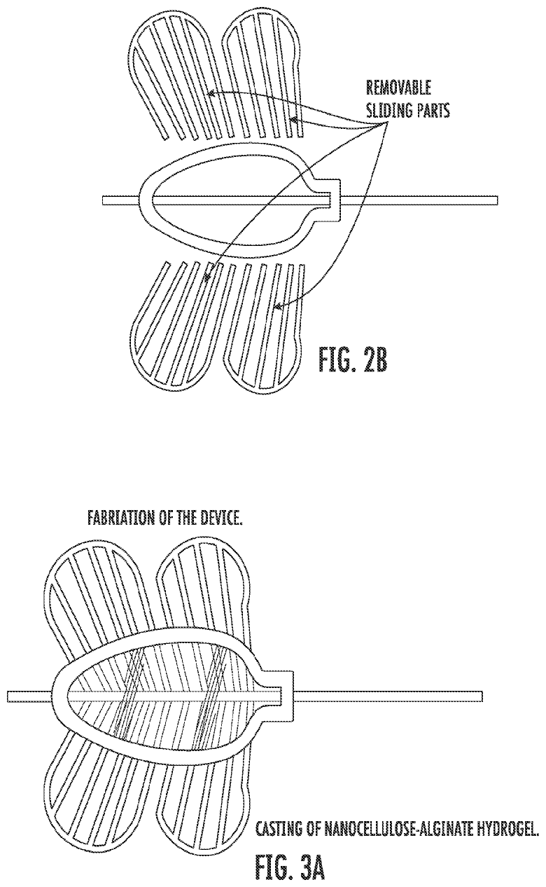

FIG. 2 shows schematically how the leaf template is constructed, in embodiments. It is composed of one main oval part and, in aspects, five (or more or less) sliding removable parts. Four (or more or less) removable parts will form branches of the vascular tree and one removable part will form the stem. FIG. 2 shows a leaf-inspired template with removable sliding parts fabricated by 3D Printing.

### Example 3 (Fabrication of the Device)

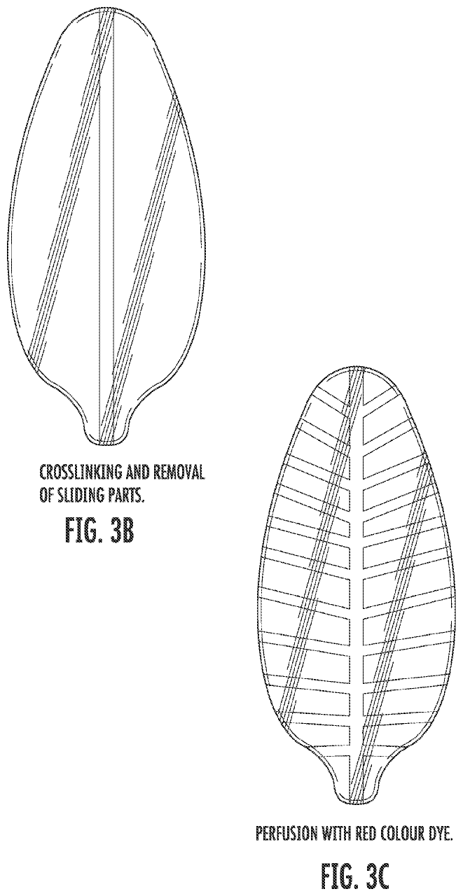

FIG. 3 visualizes a possible fabrication of the device. The first step (FIG. 3A) is a casting or injection of the matrix mixed with cells which will be delivered. Viscoelastic properties of the matrix can be selected to enable filling of the template. The bottom in an example is a petri dish. In this particular example, nanocellulose dispersion with 3% dry matter derived from tunicates (supplied by Ocean Tunicell AS, Norway) was mixed with alginate 3% solution (supplied by NovaMatrix, Norway) in a volumetric ratio of 80:20. The mixture contained 4.6% by weight of Mannitol. Selected cells can be mixed with the hydrogel after 10 minutes of crosslinking with 0.1 molar solution of Calcium chloride (the time may need to be extended if the size of the device is larger). FIG. 3B shows a crosslinked device after removal of sliding parts. FIG. 3C shows even distribution of red colour dye after perfusion through the stem and branches of the channel system inside the leaf.

### Example 4 (Leaf Loaded with Adipose Derived Stem Cells)

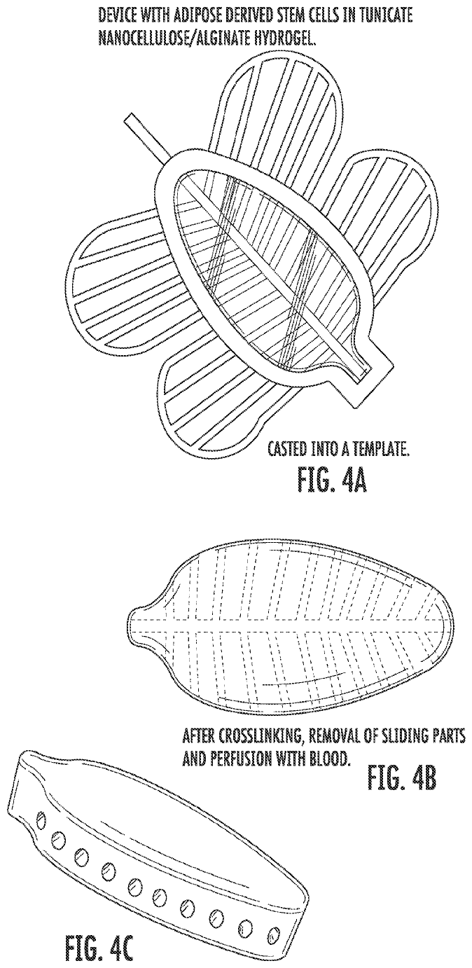

FIG. 4 shows the implantable device with adipose derived stem cells in tunicate nanocellulose/alginate hydrogel. Human lipoaspirate was processed with Lipogems device (Lipogems, Italy) to produce microfractured fat enriched with stem cells. The dispersion was mixed with nanocellulose from tunicate and alginate (ratio: 30 fat, 45 nanocellulose, 25 alginate) and casted into a leaf template. After 10 minutes of crosslinking with Calcium chloride the sliding parts were removed, and the device was perfused with blood.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compounds and/or compositions disclosed herein or with any other compounds and/or compositions. Likewise, any of the disclosed compounds and/or compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range, to the tenth of the unit disclosed, is also specifically disclosed. Any smaller range within the ranges disclosed or that can be derived from other endpoints disclosed are also specifically disclosed themselves. The upper and lower limits of disclosed ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

### REFERENCES

1. R. Fan, Y. Sun, and J. Wan, Leaf-inspired artificial microvascular networks (LIAMN) for three-dimensional cell culture, RSC Adv., 2015, 5, 90596-90601.
2. J. Priyadarshani et al., Nature-Inspired Bio-Microfluidic Device By Soft Lithography Technique, IEEE, 2018 978-1-5386-4707-3.

1. A device for delivery of cells comprising:
a crosslinked hydrogel comprising cells; and
a sliding, removable component, wherein the sliding, removable component leaves interconnected channels when removed, and wherein the interconnected channels are capable of enabling vascularization, perfusion, or combinations thereof.

2. The device according to claim 1, wherein the crosslinked hydrogel is inserted into or combined with the device by one or more of casting, injecting, pouring, or three-dimensional printing.

3. The device according to claim 1, wherein the device is made using three-dimensional printing, injection molding, or micromachining techniques.

4. The device according to claim 1, wherein the sliding, removable component is removed after the hydrogel is crosslinked.

5. The device according to claim 1, wherein the device is implanted or topologically applied to a human or animal.

6. The device according to claim 1, further comprising one or more excipients comprising one or more biocompatible materials.

7. The device according to claim 1, further comprising one or more biocompatible materials.

8. The device according to claim 1, further comprising one or more biocompatible materials comprising nanofibrillar cellulose.

9. The device according to claim 1, further comprising nanofibrillar cellulose.

10. The device according to claim 9, wherein the nanofibrillar cellulose is derived from tunicates, bacteria, and/or plants.

11. The device according to claim 1, wherein the device is used for wound healing of humans or animals.

12. The device according to claim 1, wherein the device is used to treat diabetes.

13. The device according to claim 1, wherein the device is used for drug screening.

14. The device according to claim 1, wherein the device is capable of being used in vitro or in vivo.

15. The device according to claim 1, wherein removing the sliding, removable component is capable of creating porosity with defined architecture, enabling diffusion of nutrients and oxygen, and/or contributing to survival of the cells.

16. The device according to claim 1, wherein the interconnected channels become vascularized when the device is implanted or applied.

17. The device according to claim 1, wherein the interconnected channels comprise a closed loop.

18. The device according to claim 1, wherein the interconnected channels comprise an open loop.

19. The device according to claim 1, wherein the device is capable of being perfused in vitro to act as a model of a tissue or an organ.

20. The device according to claim 1, wherein the device is used as an application to heal wounds.

21. The device according to claim 1, wherein the device is inserted or implanted to deliver stem cells or parenchymal cells.

22. The device according to claim 1, wherein the device is inserted or implanted to deliver stem cells or parenchymal cells to treat diseases, wounds, organ or tissue damage, or other medical problems or pathology.

23. A method of producing a device for delivery of therapeutic cells, comprising the following steps:
a) mixing a hydrogel with cells;
b) casting, injecting, or pouring the hydrogel mixed with cells into a three-dimensional form having a sliding removable part(s);
c) crosslinking the hydrogel; and
d) removing the sliding removable part(s), which leave interconnected channels capable of enabling vascularization and/or perfusion.

24. The method of claim 19, further comprising creating the sliding removable part(s).
